(19) United States
(12) Patent Application Publication
(10) Pub. No.: US 2015/0107601 A1
(43) Pub. Date: Apr. 23, 2015

(54) WIRELESS PATIENT POSITIONING AND WARMING DEVICE

(71) Applicant: The Curators of the University of Missouri, Columbia, MO (US)

(72) Inventors: Joshua Clay Arnone, Columbia, MO (US); Charles Matthew Sweat, Jr., Columbia, MO (US); Khushbu Jain, Columbia, MO (US); Paul S. Dale, Columbia, MO (US); Hongbin Ma, Columbia, MO (US)

(21) Appl. No.: 14/402,390

(22) PCT Filed: May 21, 2013
(86) PCT No.: PCT/US13/41997

§ 371 Date: Nov. 20, 2014

Related U.S. Application Data
(60) Provisional application No. 61/688,776, filed on May 21, 2012.

(51) Int. Cl. A61G 13/12; A61F 7/03

(52) U.S. Cl. CPC A61G 13/1275; A61F 7/03; A61G 13/127; A61F 2007/0244
USPC 128/845; 607/114

(57) ABSTRACT

The present invention provides a new and improved positioning device for stabilizing a subject on an operation table without a clinician's manual fitting. The inventive positioning device comprises a mattress/pad with a shell filled with a substance and a vacuum outlet on the sidewall of the shell. The invention also provides a warming device for preventing hypothermia of a subject on an operation table. The inventive warming device comprises a single cavity or an array of closed cuboid cavities contained in a shell and filled with a phase change material (PCM) in a form of liquid, and a triggering means for activating nucleation of the phase change material, which leads to an exothermic crystallization of the material to start the heat emission. The invention further provides a combination device that performs positioning and warming functions simultaneously.

### CROSS REFERENCE TO RELATED APPLICATIONS

This patent application claims priority to provisional U.S. patent application Ser. No. 61/688,776, filed May 21, 2012, and entitled “Wireless Patient Positioning and Warming Device”, the entire disclosure of which is expressly incorporated herein by reference.

### FIELD OF INVENTION

The present invention relates to a positioning device, a warming device, or a combination thereof, employed in a veterinary or human surgery, more specifically, to a wireless and reusable positioning and warming device to prevent hypothermia during surgery while stabilizing the position of a patient on an operating table.

### BACKGROUND OF INVENTION

Currently, during a veterinary or sometimes human surgery bean-bag vacuum positioners are used to stabilize a patient on an operation table, which requires a clinician's manual positioning to fit and stabilize the patient. At the same time, certain conductive-based and convective-based warming devices (such as a conduction heating pad) are employed to keep the patient from developing hypothermia. The current setup requires the need of external power and increases wire clutter, risk of infection, and unnecessary noises during an operation. Additionally, when a conductive heating pad is used in conjunction with a bean bag positioner, which is especially common in veterinary medicine, the heading pad counteracts the positioner's function; the stiffness of the heating pad does not allow the positioner to conform to the patient.

Therefore, there is a need to develop a new and improved positioning device which frees a clinician from manual maneuvering. There is also a need to develop a new and improved warming device which requires no external power. There is still a need to develop a new and improved combination device that is capable of performing the positioning and warming functions simultaneously.

The present invention is directed to overcoming one or more of the problems set forth above.

### SUMMARY OF INVENTION

In an aspect of the invention, a positioning device that is capable of stabilizing a patient onto an operation table without the manual fitting of a surgical clinician is disclosed. The inventive positioning device includes a mattress/pad framed with a shell wherein the shell is filled with a substance and a vacuum outlet attached to one of the sidewalls of the shell wherein a cavity/imprint of said subject is created by applying a vacuum through the vacuum outlet.

In another aspect of the invention, there is a warming device that requires no external power, employs heat emitted from an exothermic crystallization of a solution composed of a phase exchange material (PCM), and is capable of providing stable heat to prevent hypothermia of a patient during operation. The inventive warming device includes a shell; at least one cavity contained in said shell wherein the cavity is filled with a phase change material (PCM) wherein said phase change material is in a form of liquid; and a triggering means for activating nucleation of said phase change material wherein said phase change material undergoes an exothermic crystallization process upon said activation.

In another embodiment of the invention, the inventive warming device includes a single cavity contained in a shell with a top layer and a bottom layer, an array of elastic pillars connecting said top and bottom layers, a phase change material solution filled in the cavity, and a means for triggering nucleation of the phase change material solution.

According to an alternative embodiment of the invention, the inventive warming device includes an array of closed cuboid cavities contained in a shell, phase change material solution filled in the cavities, and a means for triggering nucleation of the phase change material solution in each cavity. There are multiple alternative means for triggering nucleation.

The invention further includes a combination device performing positioning and warming functions simultaneously. The inventive positioning and warming device is a multilayered structure and comprises a positioning means comprising a mattress member with a first shell filled with a substance and a vacuum outlet attached to one of the sidewalls of the first shell wherein a cavity/imprint of said subject is created by applying a vacuum through the vacuum outlet; and a warming means, attached on the top of said position component, comprising, a second shell, at least one cavity contained in said second shell wherein the cavity is filled with a phase change material (PCM) in a form of liquid, and a triggering means for activating nucleation of said phase change material wherein said phase change material undergoes an exothermic crystallization process upon said activation.

The inventive combination device may also include a first intermediate layer with a heat insulation means attached on the top of said positioning means to insulate heat generated by the warming means. The invention may further comprise a second intermediate layer with a reactivation means placed on the bottom of the warming means, whereas said reactivation means may be connected with an external power between operations to melt the crystal to its liquid form for reuse. Alternatively, an autoclave can be used to melt the crystals back into a liquid state while sterilizing the device.

These are merely some of the innumerable aspects of the present invention and should not be deemed an all-inclusive listing of the innumerable aspects associated with the present invention. These and other aspects will become apparent to those skilled in the art in light of the following disclosure and accompanying drawings.

### BRIEF DESCRIPTION OF DRAWINGS

For a better understanding of the present invention, reference may be made to the accompanying drawings in which:

FIG. 1(A) illustrates a subject placed on an exemplary positioning device;

FIG. 1(B) illustrates a cavity/imprint of the subject created on the exemplary positioning device after vacuum is applied to the device;

FIG. 2(A) is a cross-sectional illustration of an exemplary positioning device before a subject is placed on the device; FIG. 2(B) is a cross-sectional illustration of said exemplary positioning device after a cavity/imprint of a subject is created;

FIGS. 3(A) and 3(B) are the 3D and 2D illustrations of a single-cavity warming device, respectively;

FIG. 4 is a 3D illustration of an exemplary multi-cavity warming device;

FIG. 5 is an exploded illustration of a squeeze handle triggering means;

FIG. 6 is a schematic illustration of a triggering means using a nucleation trigger tab;

FIG. 7 is a 2D illustration of a heat-activating circuit of an exemplary multi-cavity warming device;

FIGS. 8(A) and (B) illustrate an exemplary individual popper employed in a triggering means for a multi-cavity warming device;

FIGS. 9(A) and 9(B) are 3D and 3D exploded illustrations of an exemplary combination device with a single-cavity warming device with a squeeze handle triggering means, respectively;

FIGS. 10(A) and 10(B) are 3D and 3D exploded illustrations of an exemplary combination device with a multi-cavity warming device with an electronic triggering means, respectively;

FIG. 11 is schematic illustration of an exemplary embodiment of a reactivating means.

Reference characters in the written specification indicate corresponding items shown throughout the drawing figures.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as to obscure the present invention.

The invention provides a new and improved positioning device for stabilizing a subject (animal or human) during an operation (such as on an operation table) with manual fitting of a surgical clinician. Unlike the current bean-bag vacuum positioners, the inventive positioning device is shaped as a mattress/pad filled with a substance such as beads. The inventive device can be constructed in various sizes for various subjects. When used in combination with the warming device (as described below), heat resistant substances can be chosen, such as silica, polypropylene, or other heat-resistant polymer beads or granules. However, it should be understood that any type of substance with sufficient level of viscosity and elasticity can be used as a substitute for beads such as polyurethane foam or the like.

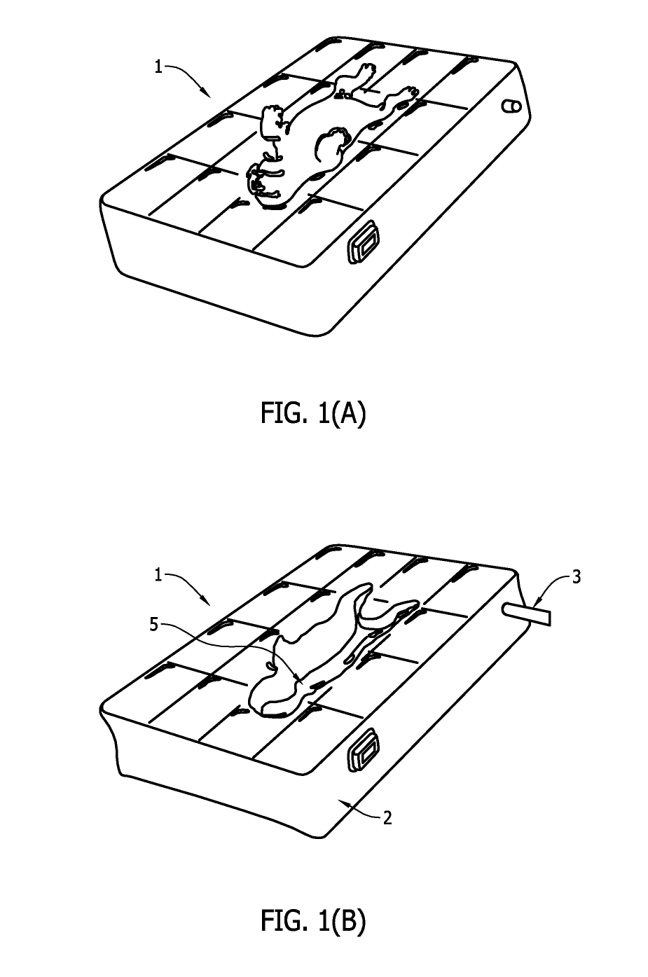

FIG. 1(A) illustrates a subject placed on an exemplary positioning device. As shown in FIG. 1(A), a subject is placed on top of an exemplary inventive positioning device. Then vacuum is applied via the vacuum valve on the sidewall of the device, so that the subject is stabilized by the shaped mattress. FIG. 1(B) illustrates a cavity/imprint of the subject created on the exemplary positioning device after vacuum is applied to the device. As shown in FIG. 1(B), a cavity/imprint 5 of the subject is created after vacuum is applied. FIG. 1(B) also shows the position device 1, shaped as a mattress/pad with a shell 2 and vacuum valve 3, on the side wall of the shell 2. The shell can comprise any type of material that can hold the substance therein. Preferably, the shell is a silicone elastomer shell. In this exemplary embodiment, the mattress/pad is filled with beads. Furthermore, any type of conventional vacuum can be applied to the exemplary positioning device.

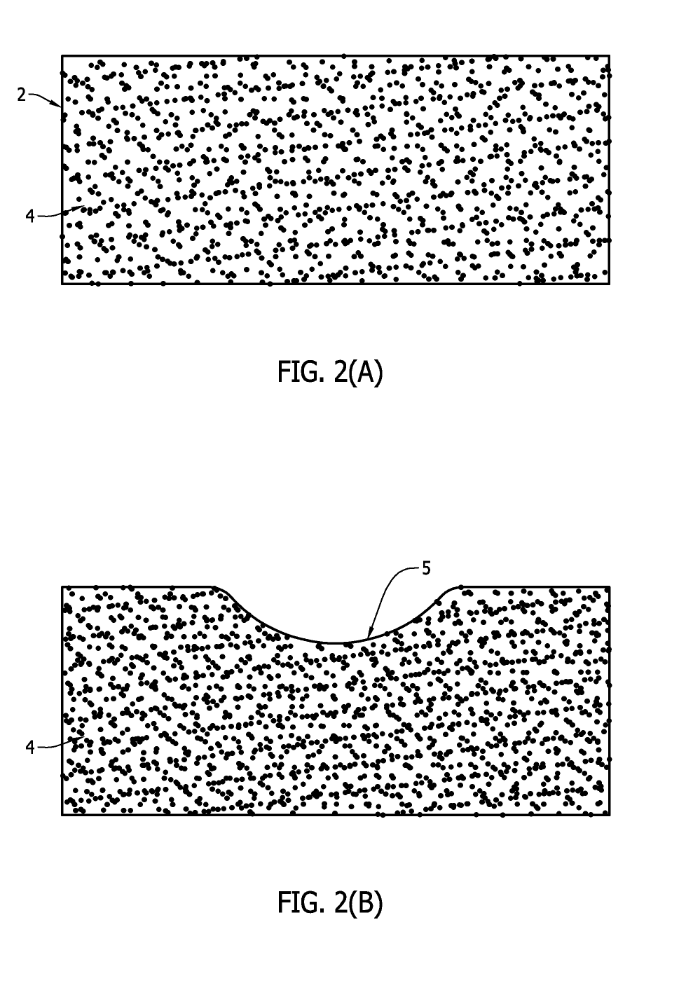

FIG. 2(A) is a cross-section view of an exemplary positioning device before a subject is placed on the device. FIG. 2(B) is a cross-sectional view of the exemplary positioning device after a cavity/imprint of a subject is created. As shown in FIG. 2(B), the weight of the subject displaced the beads 4 to form the cavity/imprint 5.

The invention also provides a new and improved warming device. While the current practice to keeping a subject warm during an operation is to lay the subject on a conductive heat pad or used forced air convection, the inventive warming device employs heat released from an exothermic crystallization of a solution. In the preferred embodiment, the warming device comprises: 1) a shell; 2) at least one cavity contained in the shell and filled with a phase change material (PCM) in a form of a solution or liquid; 3) a means for triggering nucleation of the phase change material which leads to an exothermic crystallization of the PCM solution and heat emission. The shell can comprise any type of material that can resist the heat generated by the exothermic crystallization of the PCM solution. Preferably, the shell is a silicone elastomer shell. The phase change material (PCM) is preferably a solution of sodium acetate. However, it should be understood that any other applicable phase change material can be used for purpose of generating heat.

According to one embodiment of the invention, the warming device includes a single cavity with elastic pillars connecting the upper and lower layers of the silicone elastomer shell. The elastic pillars may be arranged to prevent total displacement of sodium acetate by the weight of a subject placed on top of the warming device; in other words, the pillars provide minimal change in distance between the top and bottom silicone layers adjacent to the sodium acetate-filled cavity. The pillars may further provide minimal independent deformation between the top and the bottom silicone layers adjacent to the sodium acetate-filled cavity so that, when employed in combination with the positioning device (as described later), the positioning function of the positioning device is not negated.

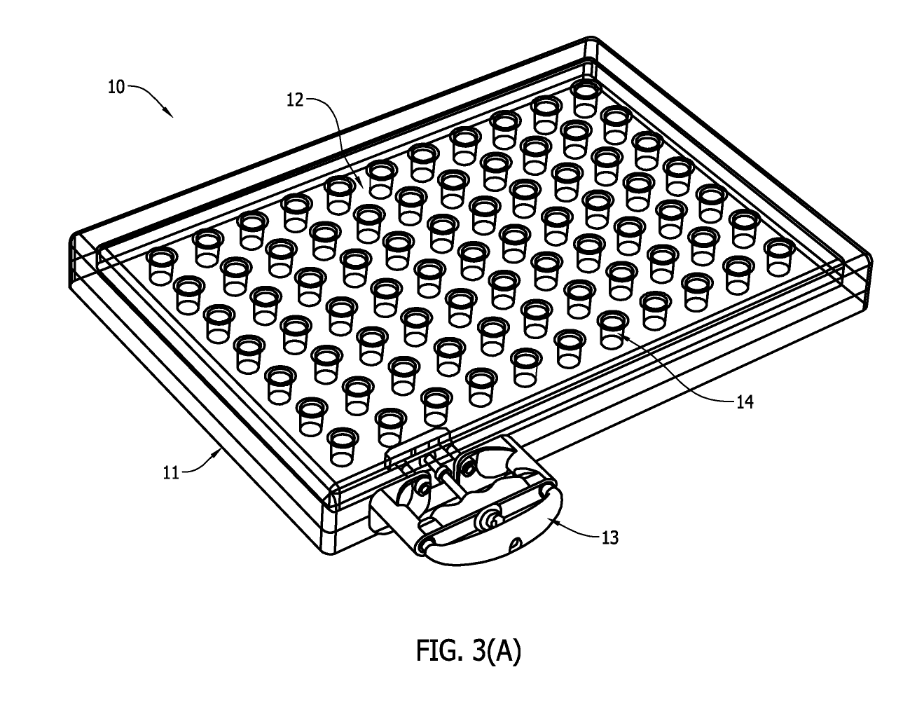

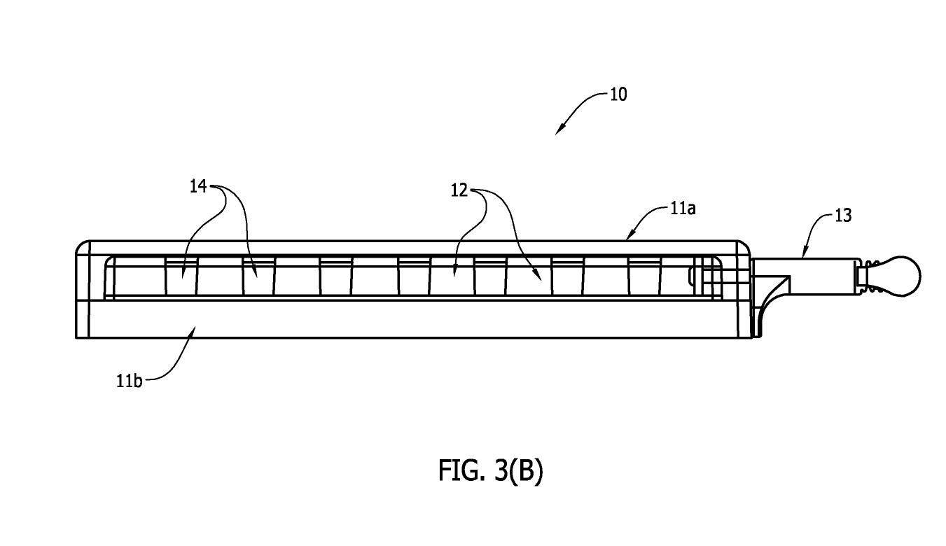

FIGS. 3(A) and 3(B) are the 3D and 2D illustrations of an exemplary embodiment of a single-cavity warming device, respectively. As shown in FIG. 3(A), the single-cavity warming device 10 comprises a silicone elastomer shell 11, a cavity 12 filled with sodium acetate contained in said shell 11, a means for starting/triggering nucleation 13, and multiple elastic pillars 14. As shown in FIG. 3(B), the multiple pillars 14 connect the upper layer 11a and the lower layer 11b of the shell 11. The multiple pillars may be in various arrangements, such as the evenly spaced array placement shown in FIGS. 3(A) and (B).

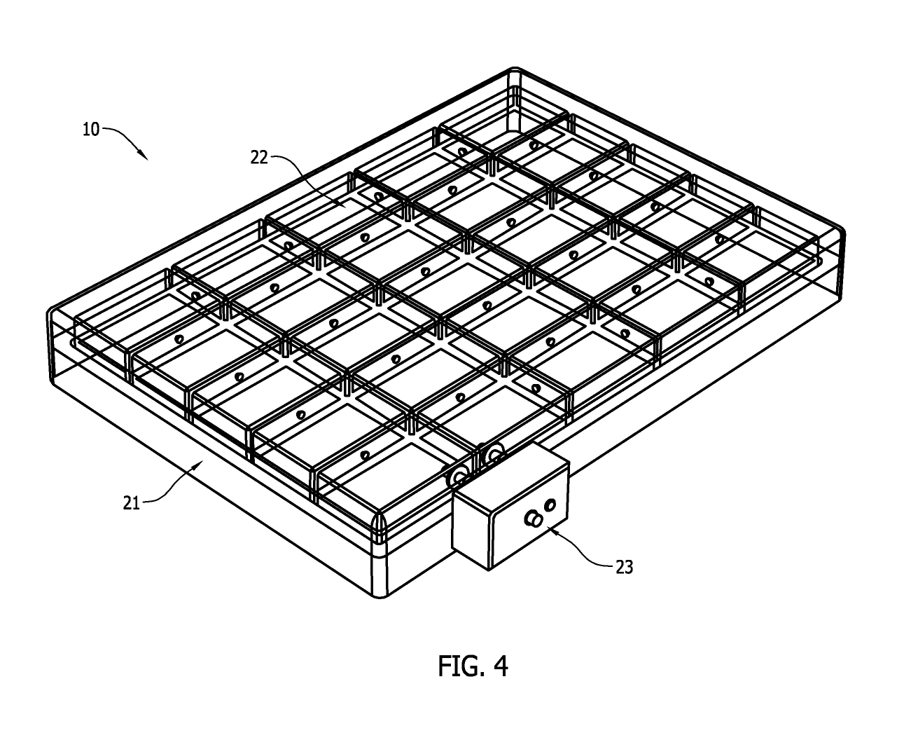

According to another embodiment of the invention, the warming device may include multiple cavities contained in the silicone elastomer shell instead of the single cavity with pillars. In this embodiment, cavities are preferably arranged in an array of closed cuboid cavities. FIG. 4 illustrates a 3D illustration of an exemplary embodiment of a multi-cavity warming device. As shown in FIG. 4, the multi-cavity warming device 20 comprises the silicone elastomer shell 21, array of cavities 22 filled with sodium acetate liquid and a means for starting/triggering nucleation 23. Preferably, but not necessarily, the sodium acetate liquid is supercooled.

There are several applicable means for triggering/starting nucleation of a phase change material. Triggering mechanism may be mechanical popping of aluminum discs or electronic activation via a closed circuit. For a single-cavity warming device, mechanical popping of aluminum disc is the preferred activation method, while for a multi-cavity warming device, mechanical popping and electronic activation can both be employed.

In a single-cavity warming device, the means for triggering nucleation can be a squeeze handle/lever/plunger outside of the shell and an aluminum disc inside of the cavity setup, whereas the squeeze motion can be transferred to the domed disc. The squeeze handle can be mounted directly onto the side wall of the shell, or make contact to the domed disc through a cable.

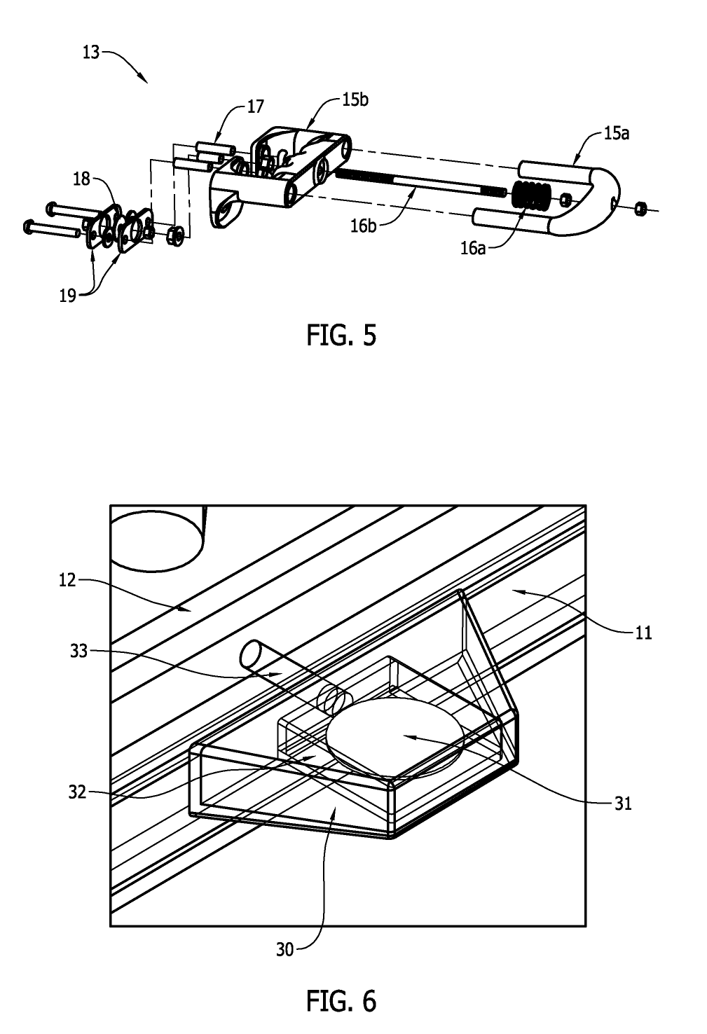

FIG. 5 is an exploded illustration of a squeeze handle triggering means. As shown in FIG. 5, the squeeze handle triggering means 13 includes an outside handle, which is made of a gripper 15a, a handle base 15b, a compression spring 16a, a partially-threaded rod 16b, whereas the gripper 15a is fitted into the handle base 15b, and the compression spring 16a threaded over the rod 16b. The outside handle can be mounted onto the side of the device shell via a pair of mounting threaded round standoffs 17. The domed disc set includes an aluminum disc 18 and a pair of stainless steel plates 19, whereas the disc 18 is loosely sandwiched between the plates 19 and mounted inside of the side wall and in alignment with the outside handle. The clearance fit between the disc and plates allows for ease of inverting, or “popping,” the aluminum disc. During an operation, the squeeze and release motion of the outside handle may pop the domed aluminum disc, which triggers the nucleation of the phase change material and starts heat emission.

Alternatively, a silicone-rubber nucleation trigger tab can be placed inside of the cavity. In this alternative embodiment, manual popping can be employed. FIG. 6 illustrates a triggering means using a nucleation trigger tab. As shown in FIG. 6, the trigger tab 30 includes a domed aluminum disc 31, a trigger cavity 32 filled with sodium acetate, and a connecting channel 33 connecting trigger cavity 32 to the main cavity 12. The trigger tab 30 can be mounted on the side wall of the shell 11, whereas the domed disc 31 is placed in the trigger cavity. The disc may be popped by hand.

Alternatively, squeeze-handle popping of the domed aluminum disc of the single cavity warming device can be replaced by DC linear actuator, pneumatic or hydraulic cylinder, or DC motor popping.

In a multi-cavity warming device, the means for triggering nucleation may be an embedded heat-activating circuit created by electrical leads, a detachable DC power supply, and a switch to electrically connect each cavity in series. When the circuit is closed, two nucleation points immediately form at the cathodes and anodes within each sodium acetate-filled cavity, leading to an exothermic crystallization of the PCM solution to start the heat emission. To achieve activation, any proper cathodes may be selected, such as, but not limited to, the cathodes described in U.S. Pat. No. 5,378,337, which is incorporated herein by reference in its entirety.

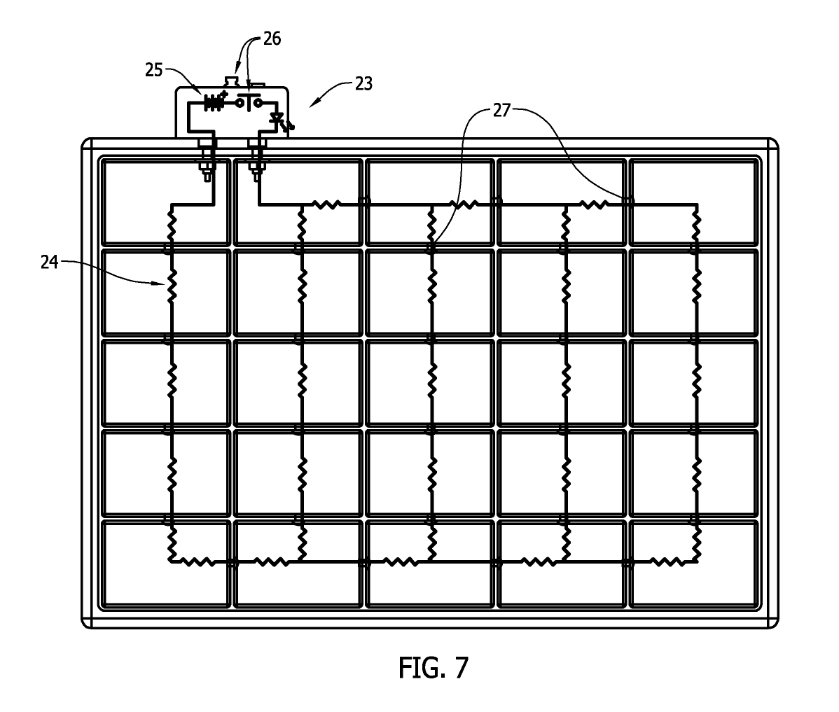

FIG. 7 is a 2D illustration of a heat-activating circuit of an exemplary multi-cavity warming device. As shown in FIG. 7, the triggering means 23 are represented as a parallel/series electrical circuit with the sodium acetate acting as resistors 24 and include a DC power supply 25, switch 26, and cathodes/anodes 27. The heat-activating circuit 24 may be closed by trigging the switch, such as a push button switch 26. All cavities are connected electronically.

In a multi-cavity warming device, a triggering mechanism may also use pneumatic or hydraulic pressure to pop aluminum discs. For example, a network of silicone-rubber tubing may be routed to each cavity, connected by T-fittings to a common pressure source, where the terminating ends are connected to a sealed fitting where within its shell is a domed disc. Applied positive or negative pressure causes the disc to pop and triggers nucleation.

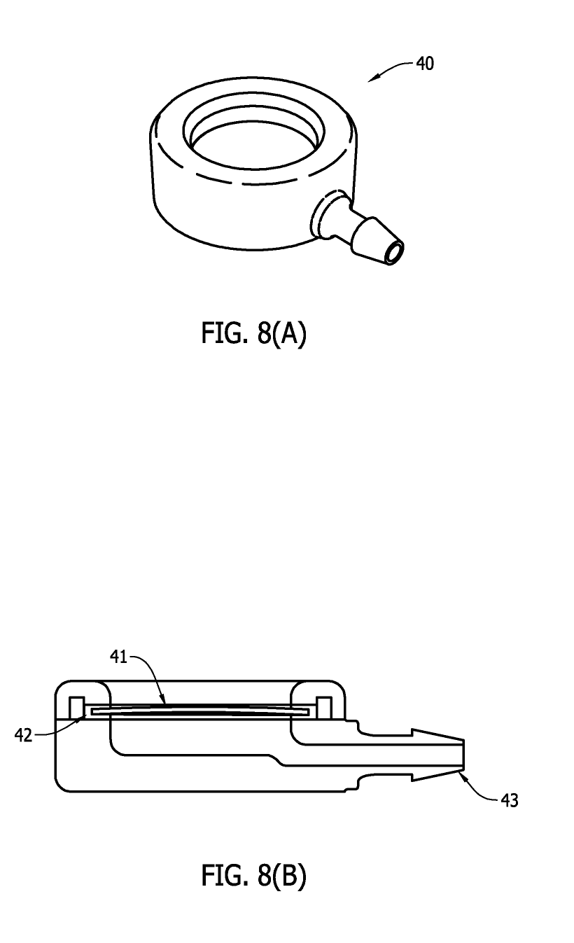

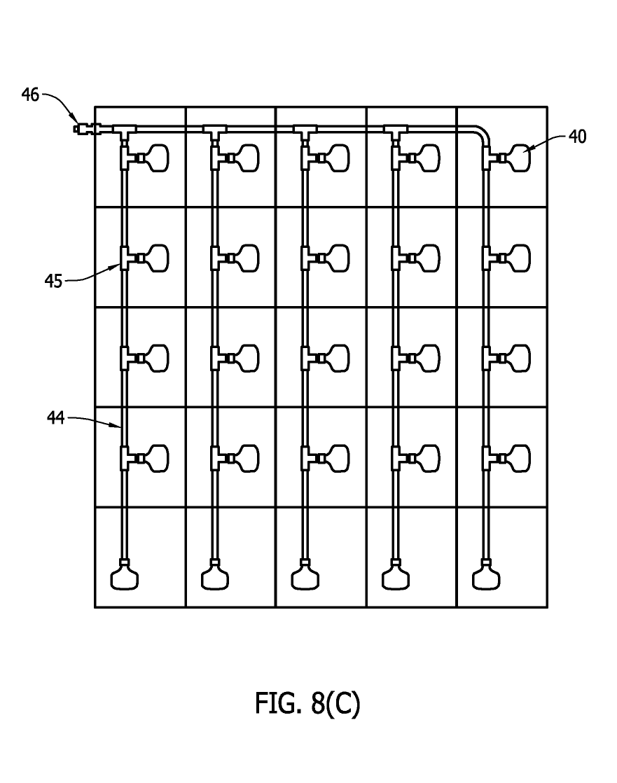

FIGS. 8(A) and 8(B) illustrate an exemplary individual popper employed in a triggering means for a multi-cavity warming device. As shown in FIGS. 8(A) and 8(B), the individual popper 40 includes a domed aluminum disc 41 sealed within a silicone-rubber adhesive sealant 42 and a flexible tube connector 43. FIG. 8(C) is a schematic illustration of the tubing layout. As shown in FIG. 8(C), the individual popper 40 in each cavity is connected to a T-fitting 45, which is connected to a heat-resistant tubing 44, which is connected to a connection point 46, which is connected to a common pressure source. The common pressure source may be a hydraulic cylinder, vacuum and pressurized air source, or portable air pump.

Though various materials may be employed as the shell of the inventive warming device, silicone elastomer rubber is chosen as the preferred material. The relatively thicker layers of silicone-rubber on the sides and below the sodium acetate liquid minimize unwanted heat loss, and the thin interfacial top layer adjacent to a subject is designed to provide an optimum conductive warming surface without burning.

A design of experiments was set up to determine the optimum depth for the sodium acetate cavities. Increasing the depth increases the amount of exothermic crystallization (and therefore increasing the duration of heat supplied as well as the temperature), but at an increase in material cost and weight. An insulated testing tray having cavities with various depths (15, 20, and 25 millimeters) was filled with the sodium acetate solution and covered with a silicone-rubber layer. After activating the crystals, the temperature of the silicone layer was measured above each cavity and compared over time. The results showed a significant difference in heating time and temperature between the 15 and 20 millimeter cavities, but little difference between the 20 and 25 millimeter cavities. Therefore, though depth of the cavity may vary, a 20 millimeter depth was chosen for the preferred design. However, it should be understood that the chosen depth of the sodium acetate solution was only intended to be an example and therefore should not be used to limit the scope of the present invention. Any other applicable depth of the sodium acetate solution can be implemented to the exemplary embodiment of the present invention.

In general, the device may provide consistent heating up to 5 hours, adequate for most operations. The warming device may be reused by melting the crystallized sodium acetate by various methods, such as autoclave, convection oven, hot water bath, heat pipe, conductive heating pad, or a sheet of Ni-Chrom heater wire. The reactivation and reuse can be repeated indefinitely as long as the solution remains within the closed system or until gradual wear over time requires a replacement.

The invention further provides a combination device capable of performing positioning and warming functions simultaneously. The combination device is equipped with a multilayer structure with a warming means at the top layer adjacent a subject and a positioning means at the bottom layer. The warming means and the positioning means are structured similarly as the warming device and positioning device described above. According to one embodiment of the invention, the combination device may also comprise an intermediate layer with a heat insulation means for insulating heat generated by the warming means from the positioning means. The heat insulation means is placed on the top of the positioning means but on the bottom of the warming means. The heat insulation means can comprise any type of material that is capable of insulating heat generated by the warming means.

According to another embodiment of the invention, the combination device may further comprise a second intermediate layer with a reactivating means for melting the sodium acetate crystal in the warming means back to its liquid form between operations. The reactivating means can be placed between the warming means and the insulation means. However it should be understood that the reactivating means can also be placed inside of the warming means. For example, the reactivating means can be placed between each pillar 14 but inside of the cavity 12 such that both top surface and bottom surface of the reactivating means can physically contact the sodium acetate filled in the cavity. This will eventually maximize the efficiency of reheating the sodium acetate crystal in the warming means.

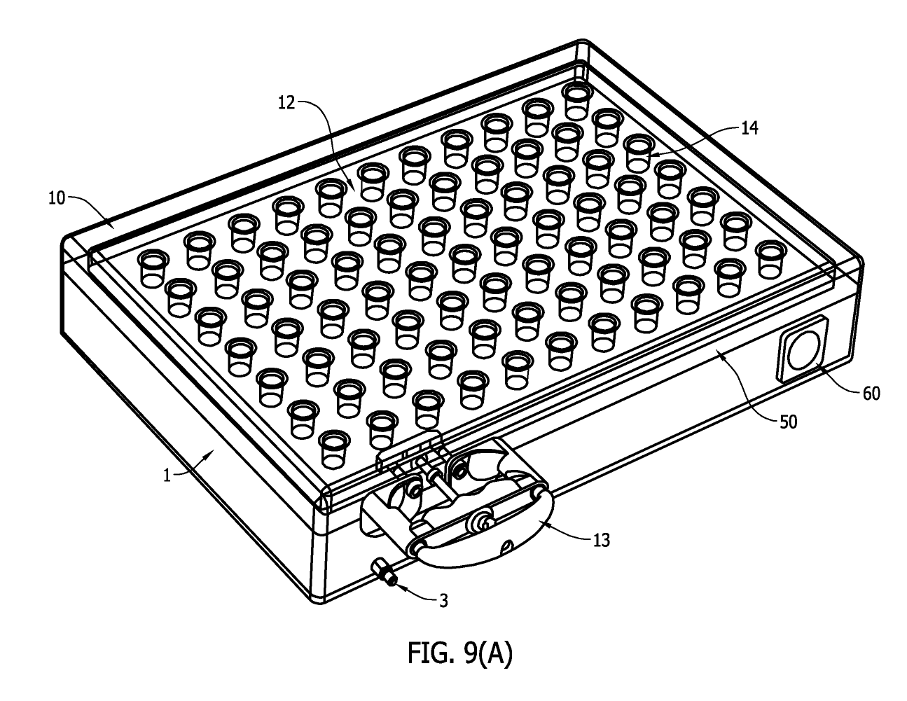

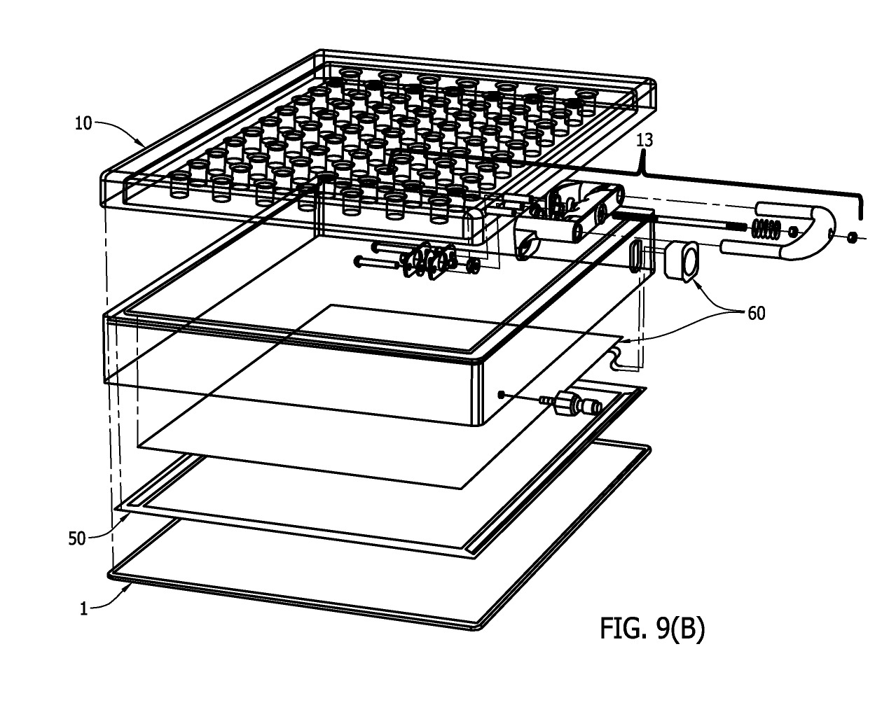

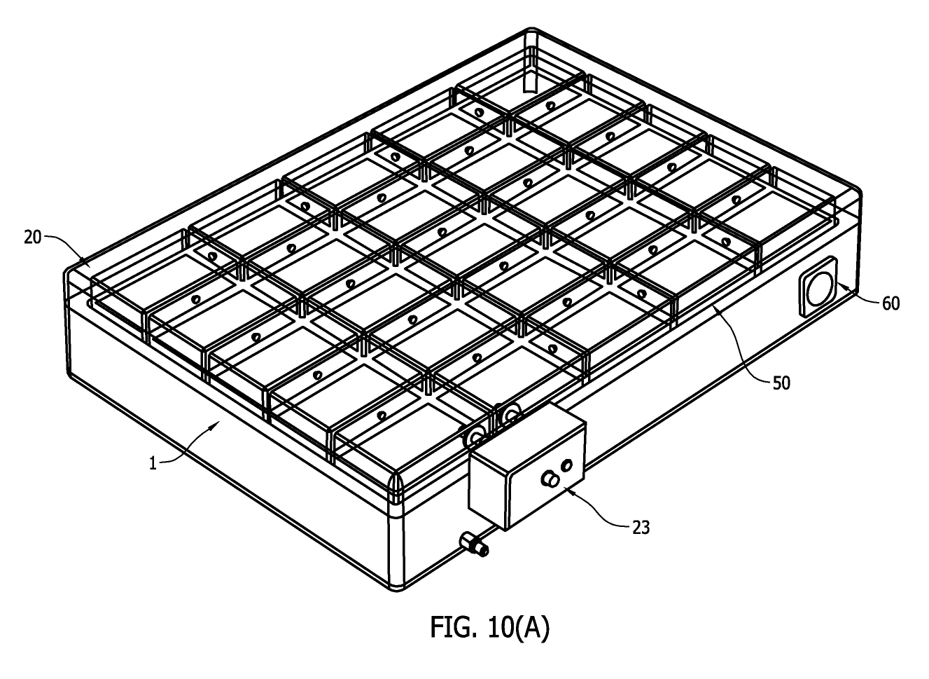

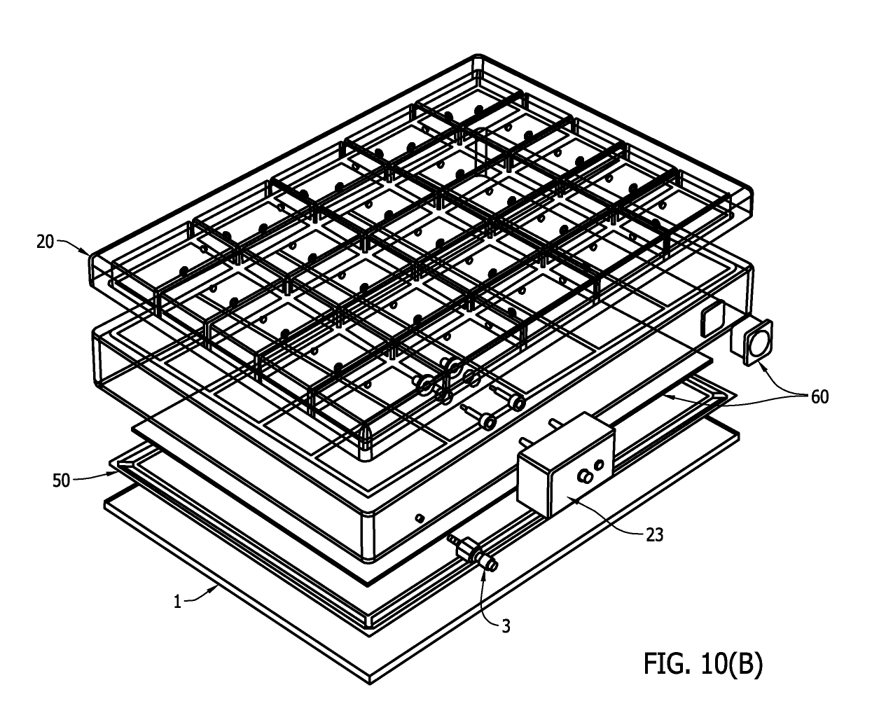

Examples of combination devices are illustrated in FIGS. 9 and 10. FIGS. 9(A) and 9(B) are 3D and 3D exploded illustrations of an exemplary combination device with a single-cavity warming device with a squeeze handle triggering means, respectively. The single-cavity warming device 10 is on the top layer, while the positioning device 1 is on the bottom layer. The reactivating means 60 and the insulation means 50 are in the middle layers. FIGS. 10(A) and 10(B) are 3D and 3D exploded illustrations of an exemplary combination device with a multi-cavity warming device with an electronic triggering means, respectively. Similarly, the reactivating means 60 and the insulation means 50 are sandwiched between the multi-cavity warming device 20 and the positioning device 1.

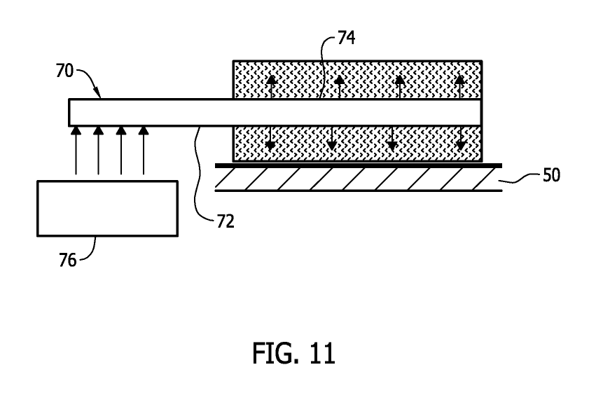

In one embodiment, the reactivating means comprises a heat pipe. As shown in FIG. 11, the heat pipe can be placed between the warming means and the insulating means 50. The heat pipe is comprised with an evaporator section 70, adiabatic section 72, and a condenser section 74. The heat pipe utilizes the evaporation heat transfer in the evaporator section 70 and condensation heat transfer in the condenser section 74, in which the vapor flows from the evaporator section 70 to the condenser section 74 is caused by the vapor pressure difference, and the liquid flow from the condenser section 74 to the evaporator section 70 is produced by either capillary force, gravitational force, electrostatic force, or other forces directly applicable to it. In this embodiment, the heat pipe preferably operates on a closed two-phase cycle and only pure liquid and vapor are present in the cycle. The working fluid preferably remains at saturation conditions as long as the operating temperature is between the triple point and the critical state.

The heat pipe can be actuated by a heat generator 76 initially. The heat generator 76 can be any conventional electrical circuit that generates heat upon the actuation. Alternatively, any type of device that generates heat may also be applied to the heat pipe of FIG. 11 or even manually applying heat to the heat pipe can be utilized. Once actuated, heat is added to the evaporator section 70 of the heat pipe and the heat is transferred through the adiabatic section 72 and reaches the condenser section 74. When the liquid in the evaporator section 70 has received enough thermal energy, the liquid vaporizes. The vapor carries the thermal energy through the adiabatic section 72 to the condenser section 74, where the vapor is condensed into the liquid and releases the latent heat of vaporization. The condensate is pumped back from the condenser section 74 to the evaporator section 70 by the driving force acting on the liquid.

The heat pipe of FIG. 11 is preferably designed to provide fast and efficient heating process to overcome low thermal conductivity of the phase change material (e.g., sodium acetate) present in the cavity 12 of FIGS. 3A, 3B, 9A, and 9B. For example, the condenser section 74 of the heat pipe of FIG. 11 is structured to maximize its surface area that generates heat such that the most of the generated heat can be transferred to sodium acetate without loss. By maximizing the surface area of the condenser section 74 that generates heat, the heat pipe can minimize the loss of the generated heat during the transfer process. Preferably, the heat pipe is in a cylinder shape, but it should be understood that the heat pipe can be made into any applicable shape. Alternatively, in order to further enhance heat transfer, fins (not shown) can be added on the condenser section 74. It should be understood that any applicable methods, other than fins, can be added on the condenser section 74 to enhance heat transfer. Alternatively, the reactivating means can be an autoclave.

Furthermore, it should be understood that when introducing elements of the present invention in the claims or in the above description of the preferred embodiment of the invention, the terms “have,” “having,” “includes” and “including” and similar terms as used in the foregoing specification are used in the sense of “optional” or “may include” and not as “required.” Similarly, the term “portion” should be construed as meaning some or all of the item or element that it qualifies.

Thus, there have been shown and described several embodiments of a novel invention. As is evident from the foregoing description, certain aspects of the present invention are not limited by the particular details of the examples illustrated herein, and it is therefore contemplated that other modifications and applications, or equivalents thereof, will occur to those skilled in the art. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims that follow.

1. A positioning device for stabilizing a subject on an operation table comprising:
a mattress/pad framed with a shell wherein the shell is filled with a substance; and
a vacuum outlet attached to one of the sidewalls of the shell, wherein a cavity/imprint of said subject is created by applying a vacuum through the vacuum outlet.

2. The positioning device in accordance with claim 1, wherein said shell is a silicone elastomer shell.

3. The positioning device in accordance with claim 1, wherein said substance comprises one of silica, polypropylene, polymer breads, and polymer granules.

4. The positioning device in accordance with claim 3, wherein said polymer breads and polymer granules are resistant to heat.

5. A warming device for keeping a subject's temperature elevated during an operation comprising:
a shell;
at least one cavity contained in said shell wherein the cavity is filled with a phase change material (PCM) wherein said phase change material is in a form of liquid; and
a triggering means for activating nucleation of said phase change material, wherein said phase change material undergoes an exothermic crystallization process upon said activation.

6. The warming device in accordance with claim 5, wherein said shell is a silicone elastomer shell.

7. The warming device in accordance with claim 5, wherein said cavity is a single cavity.

8. The warming device in accordance with claim 7, further comprising a plurality of elastic pillars wherein said elastic pillars connect the top and bottom layers of said shell.

9. The warming device in accordance with claim 5, wherein said cavity comprises multiple cavities arranged in an array formation.

10. The warming device in accordance with claim 5, wherein said phase change material (PCM) is sodium acetate.

11. The warming device in accordance with claim 10, wherein said sodium acetate is supercooled.

12. The warming device in accordance with claim 5, further comprising a reactivating means placed under said shell for melting crystalized phase change material to liquid form.

13. A combination device for stabilizing and warming a subject during an operation comprising:
a positioning means comprising a mattress member with a first shell filled with a substance and a vacuum outlet attached to one of the sidewalls of the first shell, wherein a cavity/imprint of said subject is created by applying a vacuum through the vacuum outlet; and
a warming means, attached on the top of said position component, including a second shell, at least one cavity contained in said second shell wherein the cavity is filled with a phase change material (PCM) in a form of liquid, and a triggering means for activating nucleation of said phase change material wherein said phase change material undergoes an exothermic crystallization process upon said activation.

14. The combination device in accordance with claim 13, wherein said first and second shell are a silicone elastomer shell.

15. The combination device in accordance with claim 13, wherein said substance comprises one of silica, polypropylene, polymer breads, and polymer granules.

16. The combination device in accordance with claim 15, wherein said polymer breads and polymer granules are resistant to heat.

17. The combination device in accordance with claim 13, wherein said first shell and second shell can form a single shell.

18. The combination device in accordance with claim 13, wherein said cavity is a single cavity.

19. The combination device in accordance with claim 18, further comprising a plurality of elastic pillars wherein said elastic pillars connect the top and bottom layers of said second shell.

20. The combination device in accordance with claim 13, wherein said cavity comprises multiple cavities arranged in an array formation.

21. The combination device in accordance with claim 13, wherein said phase change material (PCM) is sodium acetate.

22. The combination device in accordance with claim 21, wherein said sodium acetate is supercooled.

23. The combination device in accordance with claim 13, further comprising a heat-insulation means placed between said positioning means and said warming means for insulating heat generated by said warming means from said positioning means.

24. The combination device in accordance with claim 13, further comprising a reactivating means placed on the top of said positioning means and on the bottom of said warming means for melting said crystals to a liquid form.
